(19) United States
(12) Patent Application Publication
(10) Pub. No.: US 2025/0268669 A1
(43) Pub. Date: Aug. 28, 2025

(54) END-EFFECTORS FOR SURGICAL ROBOTIC SYSTEMS HAVING SEALED OPTICAL COMPONENTS

(71) Applicant: GLOBUS MEDICAL, INC., AUDUBON, PA (US)

(72) Inventors: James Cascarano, Cambridge, MA (US); Justin Larson, Reading, MA (US); Zachary Olenio, Derry, NH (US); Sritam Parashar Rout, Dracut, MA (US); Norbert Johnson, North Andover, MA (US)

(21) Appl. No.: 19/196,807

(22) Filed: May 2, 2025

Related U.S. Application Data
(63) Continuation of application No. 17/540,602, filed on Dec. 2, 2021, now Pat. No. 12,295,677.
Application No. 17/540,602 is a continuation of application No. 16/193,447, filed on Nov. 16, 2018, now Pat. No. 11,278,360.

(51) Int. Cl. A61B 34/30; A61B 34/20; A61B 90/00; B25J 15/00; B25J 19/02

(52) U.S. Cl. CPC A61B 34/30; B25J 15/0019; B25J 19/021; A61B 2034/2057; A61B 2034/2072; A61B 2090/3983

(57) ABSTRACT

An end-effector for a surgical robot system may include an end-effector body and an optical sub-assembly. The optical sub-assembly may include a housing coupled to the end-effector body, the housing including a threaded portion. The optical sub-assembly may further include a window that is transparent to a predetermined range of light radiation wavelengths. The optical sub-assembly may further include a gasket disposed between the housing and the window. The optical sub-assembly may further include a threaded ring disposed over the window and threadedly engaging the threaded portion of the housing, the threaded ring compressing the gasket between the window and the housing to form a seal between the window and the housing. The optical sub-assembly may further include a light emitter configured to emit light in the predetermined range of light radiation wavelengths through the window.

### CROSS-REFERENCE TO RELATED APPLICATIONS

The present application is a continuation of U.S. patent application Ser. No. 17/540,602, filed Dec. 2, 2021 and published as US 2022-0087756, which is a continuation of U.S. patent application Ser. No. 16/193,447, filed Nov. 16, 2018 and now U.S. Pat. No. 11,278,360, each of which are incorporated herein in their entirety.

### FIELD

The present disclosure relates to medical devices, and more particularly, end-effectors for surgical robotic systems having sealed optical components.

### BACKGROUND

Position recognition systems for robot assisted surgeries are used to determine the position of and track a particular object in 3-dimensions (3D). In robot assisted surgeries, for example, certain objects, such as surgical instruments, need to be tracked with a high degree of precision as the instrument is being positioned and moved by a robot or by a physician, for example.

Infrared signal based position recognition systems may use passive and/or active sensors or markers for tracking the objects. In passive sensors or markers, objects to be tracked may include passive sensors, such as reflective spherical balls, which are positioned at strategic locations on the object to be tracked. Infrared transmitters transmit a signal, and the reflective spherical balls reflect the signal to aid in determining the position of the object in 3D. In active sensors or markers, the objects to be tracked include active infrared transmitters, such as light emitting diodes (LEDs), and thus generate their own infrared signals for 3D detection.

With either active or passive tracking sensors, the system then geometrically resolves the 3-dimensional position of the active and/or passive sensors based on information from or with respect to one or more of the infrared cameras, digital signals, known locations of the active or passive sensors, distance, the time it took to receive the responsive signals, other known variables, or a combination thereof.

These surgical systems can therefore utilize position feedback to precisely guide movement of robotic arms and tools relative to a patients' surgical site.

### SUMMARY

According to some embodiments of inventive concepts, an end-effector for a surgical robot system may include an end-effector body configured to guide a surgical instrument. The end-effector may further include an optical sub-assembly coupled to the end-effector body. The optical sub-assembly may include a housing coupled to the end-effector body, the housing including an engagement portion. The optical sub-assembly may further include a window that is transparent to a predetermined range of light radiation wavelengths, the housing and the window forming a cavity. The optical sub-assembly may further include a gasket disposed between the housing and the window, the gasket defining a gasket hole therethrough. The optical sub-assembly may further include a cover portion disposed over the window and engaging the engagement portion of the housing, the cover portion compressing the gasket between the window and the housing to form a seal between the window and the housing, the cover portion defining a cover hole therethrough. The optical sub-assembly may further include a light emitter disposed in the cavity, the light emitter configured to emit light in the predetermined range of light radiation wavelengths through the gasket hole, the window, and the cover hole.

According to some other embodiments of inventive concepts, a surgical robot system is disclosed. The surgical robot system may include a robot base and an articulable robot arm coupled to the robot base. The surgical robot system may further include an end-effector coupled to the robot arm, wherein the robot arm is configured to selectively position the end-effector in a plurality of end-effector positions. The end-effector may include an end-effector body configured to guide a surgical instrument. The end-effector may further include an optical sub-assembly coupled to the end-effector body. The optical sub-assembly may include a housing coupled to the end-effector body, the housing comprising an engagement portion. The optical sub-assembly may further include a window that is transparent to a predetermined range of light radiation wavelengths, the housing and the window forming a cavity. The optical sub-assembly may further include a gasket disposed between the housing and the window, the gasket defining a gasket hole therethrough. The optical sub-assembly may further include a cover portion disposed over the window and engaging the engagement portion of the housing, the cover portion compressing the gasket between the window and the housing to form a seal between the window and the housing, the cover portion defining a cover hole therethrough. The optical sub-assembly may further include a light emitter disposed in the cavity, the light emitter configured to emit light in the predetermined range of light radiation wavelengths through the gasket hole, the window, and the cover hole. The optical sub-assembly may further include a processor circuit configured to determine, based on the light emitted by the light emitter of the optical sub-assembly, a particular one of the plurality of end-effector locations corresponding to a present location of the end-effector.

According to some other embodiments of inventive concepts, an end-effector for a surgical robot system is disclosed. The end-effector includes an end-effector body configured to guide a surgical instrument. The end-effector further includes an optical sub-assembly coupled to the end-effector body. The optical sub-assembly includes a housing coupled to the end-effector body, the housing comprising an engagement portion. The optical sub-assembly further includes a cover portion engaging the engagement portion of the housing. The optical sub-assembly further includes a gasket disposed between the housing and the cover portion, the gasket defining a gasket hole therethrough, the cover portion compressing the gasket between the cover portion and the housing to form a seal between the cover portion and the housing, the cover portion defining a cover hole therethrough. The optical sub-assembly further includes a light emitter disposed in the cavity, the light emitter configured to emit light in the predetermined range of light radiation wavelengths through the gasket hole and the cover hole.

Other methods and related surgical systems, and corresponding methods and computer program products according to embodiments of the inventive subject matter will be or become apparent to one with skill in the art upon review of the following drawings and detailed description. It is intended that all such surgical systems, and corresponding methods and computer program products be included within this description, be within the scope of the present inventive subject matter, and be protected by the accompanying claims. Moreover, it is intended that all embodiments disclosed herein can be implemented separately or combined in any way and/or combination.

### BRIEF DESCRIPTION OF THE DRAWINGS

The accompanying drawings, which are included to provide a further understanding of the disclosure and are incorporated in a constitute a part of this application, illustrate certain non-limiting embodiments of inventive concepts. In the drawings:

FIG. 1 is an overhead view of an arrangement for locations of a robotic system, patient, surgeon, and other medical personnel during a surgical procedure, according to some embodiments;

FIG. 2 illustrates a robotic system including positioning of the surgical robot and a camera relative to the patient according to some embodiments;

FIG. 3A illustrates an end-effector for use with a surgical robot system according to some embodiments;

FIG. 3B illustrates an optical component of the end-effector of FIG. 3A for use in detecting a position of the end-effector relative to a patient, according to some embodiments;

FIGS. 4A and 4B illustrate cross-sectional assembled and exploded views of a mounting arrangement for an optical component similar to the optical component of FIG. 3B in an end-effector similar to the end-effector of FIG. 3A, according to some embodiments;

FIG. 5 is an exploded view of an end-effector similar to the end-effector of FIG. 3A illustrating internal components thereof, according to some embodiments; and

FIGS. 6A and 6B illustrate cross-sectional assembled and exploded views of a windowless mounting arrangement for an optical component similar to the optical component of FIGS. 4A and 4B in an end-effector similar to the end-effector of FIG. 3A, according to some embodiments.

### DETAILED DESCRIPTION

It is to be understood that the present disclosure is not limited in its application to the details of construction and the arrangement of components set forth in the description herein or illustrated in the drawings. The teachings of the present disclosure may be used and practiced in other embodiments and practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the present disclosure. Various modifications to the illustrated embodiments will be readily apparent to those skilled in the art, and the principles herein can be applied to other embodiments and applications without departing from embodiments of the present disclosure. Thus, the embodiments are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the embodiments. Skilled artisans will recognize the examples provided herein have many useful alternatives and fall within the scope of the embodiments.

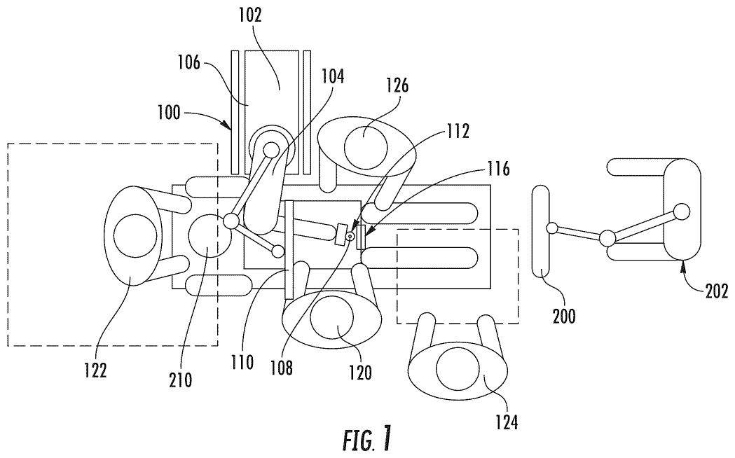

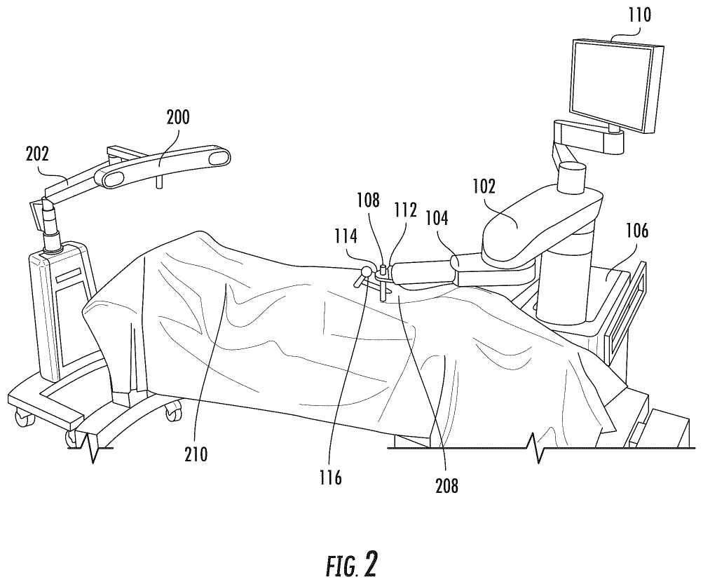

Turning now to the drawing, FIGS. 1 and 2 illustrate a surgical robot system 100 in accordance with an embodiment. Surgical robot system 100 may include, for example, a surgical robot 102, one or more robot arms 104, a base 106, a display 110, an end-effector 112, for example, including a guide tube 114, and one or more tracking markers 118. The surgical robot system 100 may include a patient tracking device 116 also including one or more tracking markers 118, which is adapted to be secured directly to the patient 210 (e.g., to a bone of the patient 210). The surgical robot system 100 may also use a sensor, such as a camera 200, for example, positioned on a camera stand 202. The camera stand 202 can have any suitable configuration to move, orient, and support the camera 200 in a desired position. The camera 200 may include any suitable camera or cameras, such as one or more infrared cameras (e.g., bifocal or stereophotogrammetric cameras), able to identify, for example, active and passive tracking markers 118 (shown as part of patient tracking device 116 in FIG. 2) in a given measurement volume viewable from the perspective of the camera 200. The camera 200 may scan the given measurement volume and detect the light that comes from the tracking markers 118 in order to identify and determine the position of the tracking markers 118 in three-dimensions. For example, active tracking markers 118 may include infrared-emitting markers that are activated by an electrical signal (e.g., infrared light emitting diodes (LEDs)), and/or passive tracking markers 118 may include retro-reflective markers that reflect infrared light (e.g., they reflect incoming IR radiation into the direction of the incoming light), for example, emitted by illuminators on the camera 200 or other suitable sensor or other device.

FIGS. 1 and 2 illustrate a potential configuration for the placement of the surgical robot system 100 in an operating room environment. For example, the robot 102 may be positioned near or next to patient 210. Although depicted near the head of the patient 210, it will be appreciated that the robot 102 can be positioned at any suitable location near the patient 210 depending on the area of the patient 210 undergoing the operation. The camera 200 may be separated from the surgical robot system 100 and positioned at the foot of patient 210. This location allows the camera 200 to have a direct visual line of sight to the surgical field 208. Again, it is contemplated that the camera 200 may be located at any suitable position having line of sight to the surgical field 208. In the configuration shown, the surgeon 120 may be positioned across from the robot 102, but is still able to manipulate the end-effector 112 and the display 110. A surgical assistant 126 may be positioned across from the surgeon 120 again with access to both the end-effector 112 and the display 110. If desired, the locations of the surgeon 120 and the assistant 126 may be reversed. The traditional areas for the anesthesiologist 122 and the nurse or scrub tech 124 may remain unimpeded by the locations of the robot 102 and camera 200.

With respect to the other components of the robot 102, the display 110 can be attached to the surgical robot 102 and in other embodiments, display 110 can be detached from surgical robot 102, either within a surgical room with the surgical robot 102, or in a remote location. End-effector 112 may be coupled to the robot arm 104 and controlled by at least one motor. In some embodiments, end-effector 112 can comprise a guide tube 114, which is able to receive and orient a surgical instrument 108 used to perform surgery on the patient 210. As used herein, the term “end-effector” is used interchangeably with the terms “end-effectuator” and “effectuator element.” Although generally shown with a guide tube 114, it will be appreciated that the end-effector 112 may be replaced with any suitable instrumentation suitable for use in surgery. In some embodiments, end-effector 112 can comprise any known structure for effecting the movement of the surgical instrument 108 in a desired manner.

The surgical robot 102 is able to control the translation and orientation of the end-effector 112. The robot 102 is able to move end-effector 112 along x-, y-, and z-axes, for example. The end-effector 112 can be configured for selective rotation about one or more of the x-, y-, and z-axis, and a Z Frame axis (such that one or more of the Euler Angles (e.g., roll, pitch, and/or yaw) associated with end-effector 112 can be selectively controlled). In some embodiments, selective control of the translation and orientation of end-effector 112 can permit performance of medical procedures with significantly improved accuracy compared to conventional robots that use, for example, a six degree of freedom robot arm comprising only rotational axes. For example, the surgical robot system 100 may be used to operate on patient 210, and robot arm 104 can be positioned above the body of patient 210, with end-effector 112 selectively angled relative to the z-axis toward the body of patient 210.

In some embodiments, the position of the surgical instrument 108 can be dynamically updated so that surgical robot 102 can be aware of the location of the surgical instrument 108 at all times during the procedure. Consequently, in some embodiments, surgical robot 102 can move the surgical instrument 108 to the desired position quickly without any further assistance from a physician (unless the physician so desires). In some further embodiments, surgical robot 102 can be configured to correct the path of the surgical instrument 108 if the surgical instrument 108 strays from the selected, preplanned trajectory. In some embodiments, surgical robot 102 can be configured to permit stoppage, modification, and/or manual control of the movement of end-effector 112 and/or the surgical instrument 108. Thus, in use, in some embodiments, a physician or other user can operate the system 100, and has the option to stop, modify, or manually control the autonomous movement of end-effector 112 and/or the surgical instrument 108. Further details of surgical robot system 100 including the control and movement of a surgical instrument 108 by surgical robot 102 can be found in co-pending U.S. patent application Ser. No. 13/924,505, which is incorporated herein by reference in its entirety.

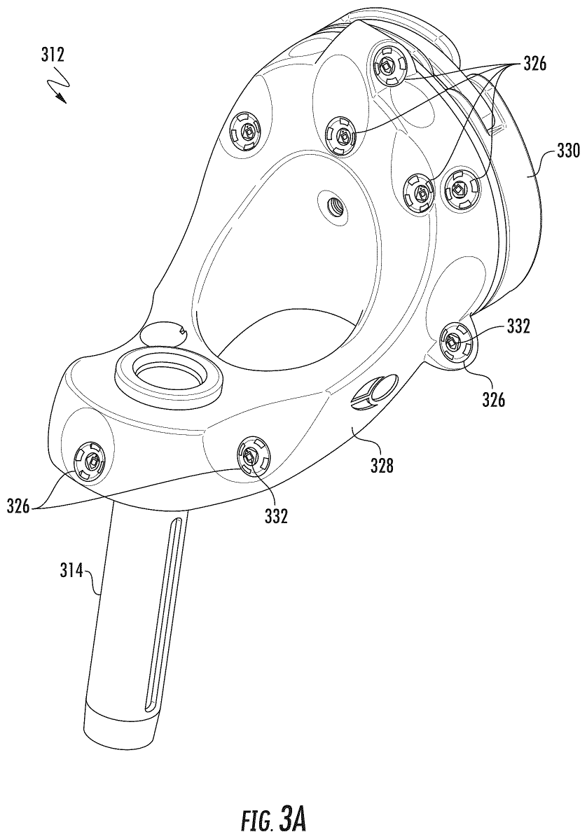

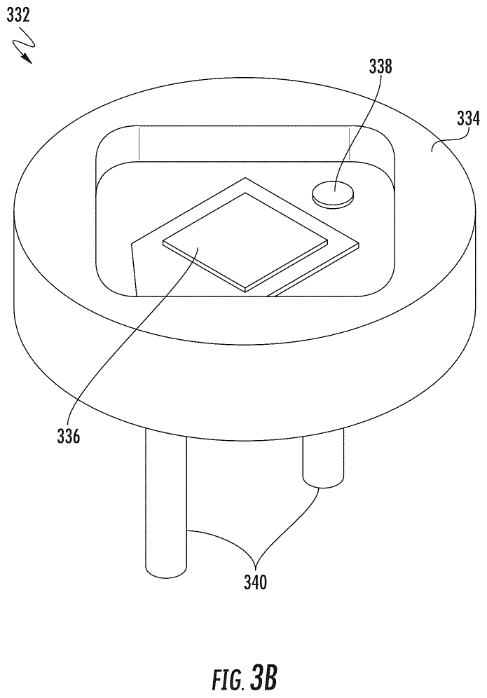

As will be described in greater detail below, the surgical robot system 100 can comprise one or more tracking markers configured to track the movement of robot arm 104, end-effector 112, patient 210, and/or the surgical instrument 108 in three dimensions. In some embodiments, a plurality of tracking markers can be mounted (or otherwise secured) thereon to an outer surface of the robot 102, such as, for example and without limitation, on base 106 of robot 102, on robot arm 104, and/or on the end-effector 112. In some embodiments, such as the embodiment of FIGS. 3A and 3B below, for example, one or more tracking markers can be mounted or otherwise secured to the end-effector 112. One or more tracking markers can further be mounted (or otherwise secured) to the patient 210. In some embodiments, the plurality of tracking markers can be positioned on the patient 210 spaced apart from the surgical field 208 to reduce the likelihood of being obscured by the surgeon, surgical tools, or other parts of the robot 102. Further, one or more tracking markers can be further mounted (or otherwise secured) to the surgical instruments 108 (e.g., a screw driver, dilator, implant inserter, or the like). Thus, the tracking markers enable each of the marked objects (e.g., the end-effector 112, the patient 210, and the surgical instruments 108) to be tracked by the robot 102. In some embodiments, system 100 can use tracking information collected from each of the marked objects to calculate the orientation and location, for example, of the end-effector 112, the surgical instrument 108 (e.g., positioned in the tube 114 of the end-effector 112), and the relative position of the patient 210. Further details of surgical robot system 100 including the control, movement and tracking of surgical robot 102 and of a surgical instrument 108 can be found in U.S. Patent Publication No. 2016/0242849, which is incorporated herein by reference in its entirety.

One drawback of conventional end-effectors is that it may be difficult to clean and sterilize the end-effector without damaging the components therein, including the markers on the end-effector and any internal electronic components of the end-effector. The robot base 106 and robot arm 104 may be isolated or shielded from the surgical environment, by a disposable plastic barrier (e.g., a cover or sleeve), for example, with the end-effector 112 attaching to an end of the robot arm 104 on the opposite side of the barrier, where the end-effector 112 may be exposed to bodily fluids, pathogens, or other contaminants during surgery. Following surgery, the end-effector 112 may be detached from the robot arm 104 for cleaning and/or sterilization, which may include a hot water soak and scrub, an ultrasonic bath, a spray rinse, and/or steam autoclave sterilization. Thus, there is a need for an end-effector design that is able to withstand these harsh conditions without sustaining damage to the end-effector or the components thereof.

Referring now to FIGS. 3A and 3B, an end-effector 312 having a plurality of tracking markers 326 for use with a surgical robot system is illustrated according to some embodiments. The end-effector 312 is similar to and may be used interchangeably with the end-effector 112 of FIGS. 1 and 2, according to some embodiments. In the embodiment of FIGS. 3A and 3B, the markers 326 include optical components 332 that include active infrared elements 336 (e.g., IR emitters and/or sensors), but it should be understood that other types of markers, such radiopaque or passive optical markers, may also be used. The markers 326 may be suitably shaped include spherical, spheroid, cylindrical, cube, cuboid, or the like. In some embodiments, one or more of markers 326 may be optical markers. In some embodiments, the positioning of one or more tracking markers 326 on end-effector 312 can maximize the accuracy of the positional measurements by serving to check or verify the position of end-effector 312.

Embodiments include one or more markers 326 coupled to the surgical instrument 108. In some embodiments, these markers 326, can comprise conventional infrared light-emitting diodes (LEDs) or an Optotrak® diode capable of being tracked using a commercially available infrared optical tracking system such as Optotrak®. Optotrak® is a registered trademark of Northern Digital Inc., Waterloo, Ontario, Canada. In other embodiments, markers 326 can comprise conventional reflective spheres capable of being tracked using a commercially available optical tracking system such as Polaris Spectra®. Polaris Spectra® is also a registered trademark of Northern Digital, Inc.

As discussed above, the markers 326 coupled to the end-effector 312 in this embodiment include active IR elements 336, which employ infrared light-emitting diodes which may be turned on and off. In some embodiments, light emitted from markers 326 (as well as light emitted or reflected from other markers, such as passive markers on a patient or instrument) can be detected by a camera, such as the camera 200 of FIGS. 1 and 2 for example, and can be used to monitor the location and movement of the end-effector 312 and other marked objects. In some alternative embodiments, markers 326 can include a radio-frequency and/or electromagnetic emitter, reflector and/or transceiver and the camera can include or be replaced by a radio-frequency and/or electromagnetic receiver and/or transceiver. In many embodiments, a camera or other end-effector sensor may detect the light emitted by the IR emitter and generate a location signal containing location information for the IR emitter.

As discussed above, the end-effector 312 may be exposed to harsh conditions during cleaning and/or sterilization, including extreme heat and/or moisture. One method of sealing and isolating the internal components of end-effectors includes disposing a potting material around the internal electronic components of the end-effector, such as the IR elements 336 of the markers 326, internal printed circuit assembly (PCA) (not shown), or other electronic components. The potting material may expand when exposed to heat, which may enhance the sealing properties of the potting material. However, this expansion may also result in physical damage to the internal components, such as solder joint fractures, for example, which may be compressed by the expanding potting material.

In the embodiment of FIGS. 3A and 3B, the end-effector 312 includes a body 328 that forms a guide tube 314 for retaining and/or orienting a surgical instrument, such as the surgical instrument 108 of FIGS. 1 and 2, for example. The end-effector 312 of FIGS. 3A and 3B also includes a mounting collar 330 for selectively coupling the end-effector 312 to a robot arm, such as the robot arm 104 of FIGS. 1 and 2, for example. Each of the plurality of optical components 332 includes an optical component housing 334, which may be separately formed from the end-effector body 328, or may be integrally formed with the end-effector body 328, as desired. Referring now to FIG. 3B, in addition or as an alternative to the IR element 336, which may allow the optical component 332 to emit IR light or detect or sense IR light being emitted by a different IR light source for example, the optical component 332 may also or alternatively includes one or more contact elements 338 (or other type of element), which may allow additional components to be connected to the optical component 332. The optical component 332 may also include electrical leads 340 for providing electrical power to and/or electrical signals to or from the optical component.

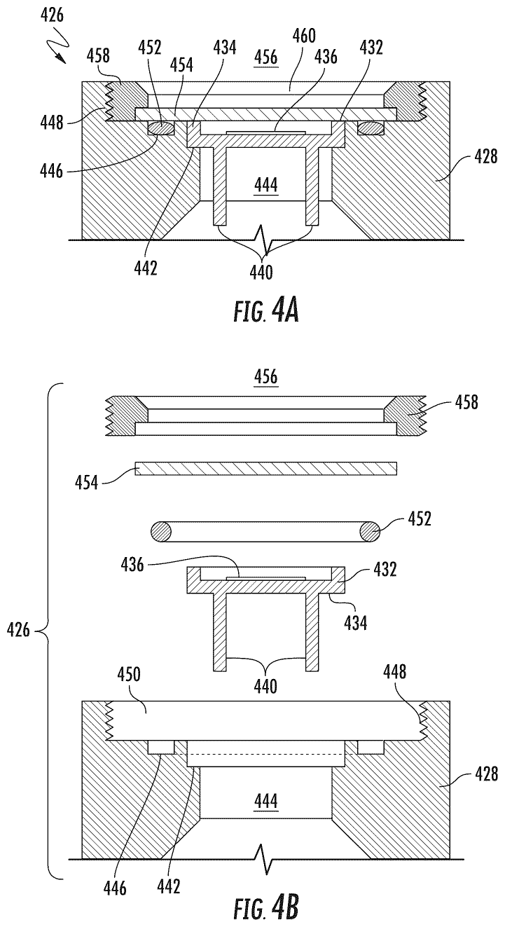

Referring now to FIGS. 4A and 4B, cross-sectional assembled and exploded views of a mounting arrangement for tracking marker 426 similar to the optical component 332 of FIG. 3B in an end-effector similar to the end-effector 312 of FIG. 3A is illustrated, according to some embodiments. The body 428 includes an optical component seat 442 for receiving and engaging the optical component housing 434 so that the leads 440 of the optical component 432 are disposed on an interior cavity 444 in the body 428. The body 428 of the tracking marker 426 also includes an O-ring seat 446, which is an annular cylindrical groove in this example. The body 430 of the tracking marker 426 also includes a threaded engagement portion 448, which includes inwardly facing threads to form a cylindrical recess 450 in the body 428 of the tracking marker 426 in this example.

To assemble the tracking marker 426, the optical component housing 434 is disposed in the component seat 442 of the body 428 of the tracking marker 426, and a compressible O-ring 452 is disposed in the O-ring seat 446. The O-ring 452 may be formed from any suitable material, such as rubber or another flexible or semi-flexible material, based on the material's ability to form a waterproof and/or moisture-proof seal, to withstand extreme temperatures, chemical exposure, or other properties of sterilization techniques, such as steam autoclave, hydrogen peroxide plasma or vapor, or ethylene oxide, for example. The O-ring 452 operates as a gasket in this embodiment, and it should be understood that other types of gaskets or other components may be used for forming an appropriate seal, as desired.

A sapphire window 454 is disposed over the O-ring 452 and the optical component 432 to separate the interior cavity 444 of the body 428 from an exterior side 456 of the body 428. A threaded cover portion 458 that is complementary to the threaded engagement portion 448 of the body 428 is screwed into the cylindrical recess 450 to engage and compress the sapphire window against the compressible O-ring 452, thereby forming a moisture-proof seal between the interior cavity 444 of the body 428, which contains the IR element 436, leads 440 and other components of the optical component 432, and the exterior side 456 of the body 428. It should be understood that, while the threaded cover portion 458 and the threaded engagement portion 448 threadedly engage each other to form a moisture proof seal, other types of engagement portions, cover portions, and mechanisms may be used. The threaded cover portion 458 forms an aperture 460 over the sapphire window 454 that allows the IR element 436 to emit light toward the exterior side 456 of the body 428 through a gasket hole formed by the O-ring 452, the sapphire window 454, and a cover hole formed by the threaded cover portion 458. In this embodiment, the thin (e.g., 1 mm or less) window 454 is formed from sapphire because of its hardness and durability, and its transparency to IR light, but it should be understood that other materials may be used, as desired. For example, borosilicate may be another suitable material having similar optical properties and biocompatibility to sapphire. In this example, the IR emitter is configured to configured to emit light within a wavelength range of 700 nanometers to 1 millimeter, i.e., the IR wavelength band, and the sapphire window 454 is transparent to some or all of the same IR wavelength band. It should be understood, however, that other light emitters that are configured to emit light in a different predetermined range of light radiation wavelengths may be used.

In this example, the seal formed by the threaded cover portion 458 compressing the O-ring 452 between the sapphire window 454 and the optical component housing 434 forms a cavity seal that atmospherically isolates the cavity 444 from an atmosphere outside the end-effector, i.e., the exterior side 456 of the body 428, while also allowing the cavity 444 to be devoid of potting material. While the threaded cover portion 458 includes outwardly-facing threads that engage with complementary interior-facing threads of the threaded engagement portion 448 of the body 428 in this example, it should be understood that other arrangements are contemplated. For example, a threaded cover portion having inwardly-facing threads may engage with complementary exterior-facing threads of a threaded engagement portion of the body, in some embodiments. Other arrangements may include, in addition or as an alternative, adhesives, friction fit, fasteners, or other arrangements, as desired. In this example, as well, the threaded cover portion 458 includes a chamfered (e.g., 120 degree) interior surface so that the threaded cover portion 458 does not block the visibility of the IR emitter to an end-effector sensor, such as a camera.

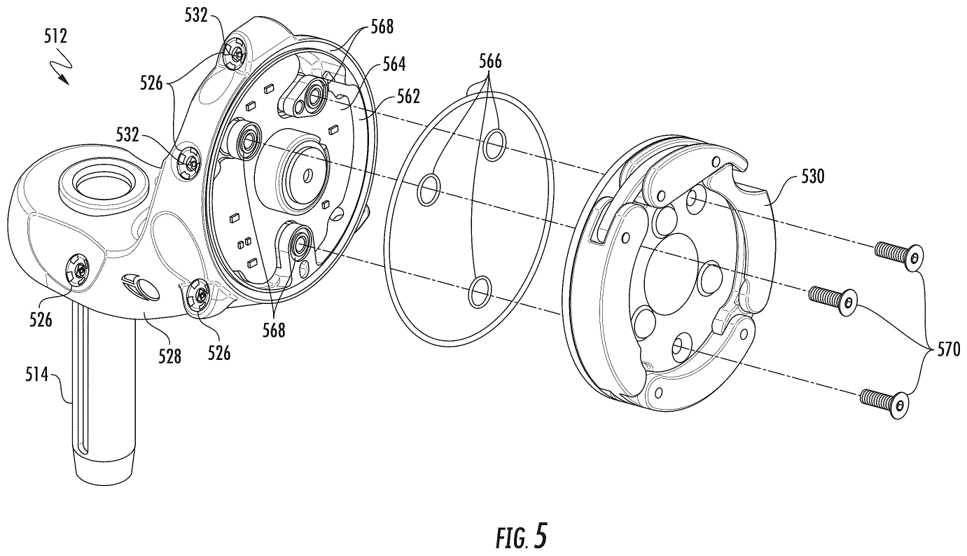

Referring now to FIG. 5, an exploded view of an end-effector 512 similar to the end-effector 312 of FIG. 3A is illustrated, according to some embodiments. The end-effector 512 in this embodiment forms a guide tube 514 for retaining and/or orienting a surgical instrument, such as the surgical instrument 108 of FIGS. 1 and 2, for example, and also includes a plurality of tracking markers 526 similar to the tracking marker 326, 426 of FIGS. 3A-4B, for example. The end-effector body 528 forms an interior portion 562, with electronic components 564, such as a PCB or other circuitry for controlling the tracking markers 526 and/or other components of the end-effector 512. In this embodiment, a plurality of compressible O-rings 566 are seated in a plurality of complementary O-ring seats 568. The mounting collar 530 is secured to the body 528 of the end-effector 512 to compress the compressible O-rings 566 therebetween, thereby forming moisture-proof seals between the mounting collar 530 and the body 528. In this manner, the entire interior portion 562 of the end-effector 512 may be sealed, thereby protecting the optical components 532 of the tracking markers 526 and the other electronic components 564 of the end-effector 512.

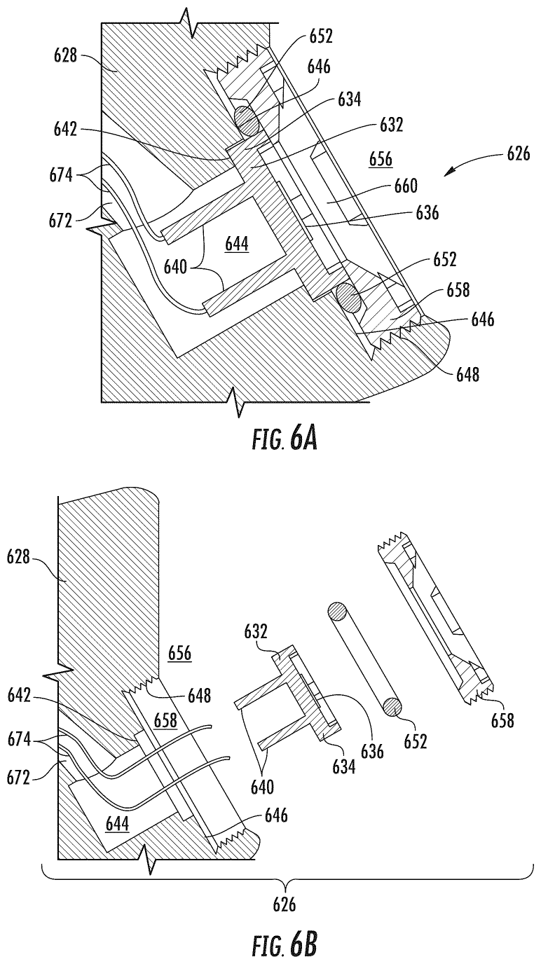

The tracking markers described above, such as the tracking marker 326 of FIGS. 3A and 3B for example, may include a sapphire window that separates the optical component of the tracking marker from an outside environment. It should be understood, however, that a sapphire window or other window may be omitted in some embodiments. For example, in some embodiments, the exterior facing portions of the optical component may be sufficiently durable that a protective window is not necessary. In this regard, FIGS. 6A and 6B illustrate cross-sectional assembled and exploded views of a windowless mounting arrangement for an optical component 632 similar to the optical component of FIGS. 4A and 4B in an end-effector similar to the end-effector 312 of FIG. 3A, according to some embodiments.

In this example, the body 628 includes an optical component seat 642 for receiving and engaging the optical component housing 634 so that the leads 640 of the optical component 632 are disposed on an interior side 644 of the body 628. In this example, the optical component housing 634 includes an O-ring seat 646 for receiving a compressible O-ring 652. The body 630 of the tracking marker 626 also includes a threaded engagement portion 648, which includes inwardly facing threads to form a cylindrical recess 650 in the body 628 of the tracking marker 626 in this example.

To assemble the tracking marker 626, the optical component housing 634 is disposed in the component seat 642 of the body 628 of the tracking marker 626, and a compressible O-ring 652 is disposed around the O-ring seat 646 of the optical component housing 634. A threaded cover portion 658 that is complementary to the threaded engagement portion 648 of the body 628 is screwed into the cylindrical recess 650 to engage and compress the O-ring 652, thereby forming a moisture-proof seal between the interior side 644 of the body 628, which contains the leads 640 and other components of the optical component 632, and the exterior side 656 of the body 628. The threaded cover portion 658 forms an aperture 660 that allows the IR element 636 to emit light toward the exterior side 656 of the body 628. In this embodiment, the IR element 636 may be sufficiently durable that the IR element 636 may be exposed to an exterior environment during use, cleaning and/or sterilization of the end-effector, which may allow for a simplified windowless arrangement, such as the arrangement of FIGS. 6A and 6B.

In the above-description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belong. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

When an element is referred to as being “connected”, “coupled”, “responsive”, or variants thereof to another element, it can be directly connected, coupled, or responsive to the other element or intervening elements may be present. In contrast, when an element is referred to as being “directly connected”, “directly coupled”, “directly responsive”, or variants thereof to another element, there are no intervening elements present. Like numbers refer to like elements throughout. Furthermore, “coupled”, “connected”, “responsive”, or variants thereof as used herein may include wirelessly coupled, connected, or responsive. As used herein, the singular forms “a”, “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus a first element/operation in some embodiments could be termed a second element/operation in other embodiments without departing from the teachings of present inventive concepts. The same reference numerals or the same reference designators denote the same or similar elements throughout the specification.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processor circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s). For example, in some embodiments, a processor circuit may be configured to determine, based on light emitted by one or more light emitters of an optical sub-assembly of an end-effector, a particular one of the plurality of end-effector positions corresponding to a present location of the end-effector. In some embodiments, the wherein the processor circuit is configured to determine a particular one of a plurality of end-effector positions based on the location signal.

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Although several embodiments of inventive concepts have been disclosed in the foregoing specification, it is understood that many modifications and other embodiments of inventive concepts will come to mind to which inventive concepts pertain, having the benefit of teachings presented in the foregoing description and associated drawings. It is thus understood that inventive concepts are not limited to the specific embodiments disclosed hereinabove, and that many modifications and other embodiments are intended to be included within the scope of the appended claims. It is further envisioned that features from one embodiment may be combined or used with the features from a different embodiment(s) described herein. Moreover, although specific terms are employed herein, as well as in the claims which follow, they are used only in a generic and descriptive sense, and not for the purposes of limiting the described inventive concepts, nor the claims which follow. The entire disclosure of each patent and patent publication cited herein is incorporated by reference herein in its entirety, as if each such patent or publication were individually incorporated by reference herein. Various features and/or potential advantages of inventive concepts are set forth in the following claims.

1. An end-effector for a surgical robot system, comprising:
an end-effector body configured to guide a surgical instrument and adapted to attach to a robotic arm, the end effector including:
an optical sub-assembly received in the end-effector body and including:
an optical component housing configured to be received by an optical component seat of the end-effector body;
an O-ring disposed in an O-ring seat of the optical component housing;
a threaded cover portion configured to engage with a threaded engagement portion of the end-effector body and compress the O-ring to form a seal between an interior side of the end-effector body and an exterior side of the end-effector body; and
a tracking marker disposed in an internal cavity of the end-effector body, the tracking marker configured to be tracked by a navigation camera system.

2. The end-effector of claim 1, wherein the optical sub-assembly comprises an infrared (IR) emitter.

3. The end-effector of claim 1, wherein the optical sub-assembly comprises a plurality of optical sub-assemblies received in a plurality of recesses in the end effector body.

4. The end-effector of claim 1, wherein the seal formed by the threaded cover portion compressing the O-ring between an interior side of the end-effector body and an exterior side of the end-effector body is a moisture-proof seal.

5. The end-effector of claim 1, wherein the seal formed by the threaded cover portion compressing the O-ring between an interior side of the end-effector body and an exterior side of the end-effector body forms a cavity seal that atmospherically isolates the internal cavity from an atmosphere outside the end-effector.

6. The end-effector of claim 1, wherein:
the end effector body includes an inwardly facing thread; and
the threaded cover portion includes an outwardly facing thread that is complementary to the inwardly facing thread of the end effector body.

7. The end-effector of claim 1, wherein:
the threaded cover portion includes an inwardly facing thread, and
the end effector body includes an outwardly facing thread that is complementary to the inwardly facing thread of the retainer cover.

8. The end-effector of claim 1, wherein the end-effector body includes a tube through which the surgical instrument is received.

9. An tracking marker for an end-effector of a surgical robot system, the tracking marker comprising:
a body comprising an optical component seat;
an optical component housing configured to be received by the optical component seat;
an O-ring disposed in an O-ring seat of the optical component housing;
a threaded cover portion configured to engage with a threaded engagement portion of the body and compress the O-ring to form a seal between an interior side of the body and an exterior side of the body; and
a tracking marker disposed in an internal cavity of the body, the tracking marker configured to be tracked by a navigation camera system.

10. The tracking marker of claim 9, wherein the optical sub-assembly comprises an infrared (IR) emitter.

11. The tracking marker of claim 9, wherein the optical sub-assembly comprises a plurality of optical sub-assemblies received in a plurality of recesses in the end effector body.

12. The tracking marker of claim 9, wherein the seal formed by the threaded cover portion compressing the O-ring between an interior side of the body and an exterior side of the body is a moisture-proof seal.

13. The tracking marker of claim 9, wherein the seal formed by the threaded cover portion compressing the O-ring between an interior side of the body and an exterior side of the body forms a cavity seal that atmospherically isolates the internal cavity from an atmosphere outside the end-effector.

14. The tracking marker of claim 9, wherein:
the end effector body includes an inwardly facing thread; and
the threaded cover portion includes an outwardly facing thread that is complementary to the inwardly facing thread of the end effector body.

15. The end-effector of claim 9, wherein:
the threaded cover portion includes an inwardly facing thread, and
the end effector body includes an outwardly facing thread that is complementary to the inwardly facing thread of the retainer cover.

16. The end-effector of claim 9, wherein the end-effector body includes a tube through which the surgical instrument is received.
